(19) United States
(12) Patent Application Publication
(10) Pub. No.: US 2014/0039035 A1
(43) Pub. Date: Feb. 6, 2014

(54) COMPOSITIONS AND METHODS FOR TREATING OBESITY

(71) Applicants: Sheng Bi, Woodstock, MD (US); Timothy Moran, Towson, MD (US)

(72) Inventors: Sheng Bi, Woodstock, MD (US); Timothy Moran, Towson, MD (US)

(73) Assignee: The Johns Hopkins University, Baltimore, MD (US)

(21) Appl. No.: 13/988,095

(22) PCT Filed: Nov. 18, 2011
(86) PCT No.: PCT/US2011/061385

§ 371 Date: Oct. 3, 2013

Related U.S. Application Data
(60) Provisional application No. 61/415,080, filed on Nov. 18, 2010.

(51) Int. Cl. C12N 15/113

(52) U.S. Cl. CPC C12N 15/113
USPC 514/44 A; 536/23.1; 435/320.1; 435/369

(57) ABSTRACT

The present invention relates to the field of obesity. More specifically, the present invention provides methods and compositions useful in treating obesity and obesity-associated conditions. In a specific embodiment, a method for treating obesity comprises administering an effective amount of an agent that inhib its expression of neuropeptide Y (NPY). In another embodiment, the present invention provides a recombinant nucleic acid construct comprising a nucleic acid sequence encoding an oligonucleic acid, wherein the oligonucleic acid comprises at least one sequence substantially complementary to at least a part of the neuropeptide Y (NPY) gene or transcript thereof, and wherein the oligonucleic acid inhibits or reduces the expression of NPY.

### CROSS-REFERENCE TO RELATED APPLICATIONS

This application claims the benefit of U.S. Provisional Application No. 61/415,080, filed Nov. 18, 2010; which is incorporated herein by reference in its entirety.

### STATEMENT OF GOVERNMENTAL INTEREST

This invention was made with U.S. government support under grant no. DK74269. The U.S. government has certain rights in the invention.

### FIELD OF THE INVENTION

The present invention relates to the field of obesity. More specifically, the present invention provides methods and compositions useful in treating obesity and obesity-associated conditions.

### BACKGROUND OF THE INVENTION

Obesity has become a global epidemic afflicting both children and adults, and gradually spreading from the Western countries to the developing nations as well. It is now widely recognized that obesity is associated with, and is actually a major culprit in numerous comorbidities such as cardiovascular diseases (CVD), type 2 diabetes, hypertension, certain cancers, and sleep apnea/sleep-disordered breathing. As recently acknowledged by a joint American Heart Association and American Diabetes Association (AHA/ADA) statement, obesity is an independent risk factor for CVD, and CVD risks have also been documented in obese children. Obesity is associated with an increased risk of overall morbidity and mortality as well as reduced life expectancy. Indeed, obesity and overweight are now listed as independent cardiovascular risk factors in the joint AHA/ADA call for the prevention of cardiovascular disease and diabetes.

With the exception of bariatric surgery, which can only be offered to a limited number of subjects, the lack of any truly effective treatment for obesity highlights the gravity of current prospects to control the obesity epidemic. Preventive measures have generally failed; effective public and political strategies to reshape lifestyle by proper nutrition and exercise so as to counteract the global obesity trends have not yet been formulated. Finally, the current generation of weight-reducing medications offers limited benefit, and indeed, despite more than a decade of use has failed to impact the global obesity challenge. Health service use and medical costs associated with obesity and related diseases have risen dramatically and are expected to continue to rise. Accordingly, novel therapeutic strategies to combat obesity are needed.

### SUMMARY OF THE INVENTION

The present invention is based, at least in part, on the discovery that neuropeptide Y (NPY) plays a critical role in body weight regulation. Knockdown of NPY expression in the dorsomedial hypothalamus (DMH) by adeno-associated virus-mediated RNAi reduced fat depots in rats fed regular chow and ameliorated high-fat diet-induced hyperphagia and obesity. DMH NPY knockdown also resulted in development of brown adipocytes in inguinal white adipose tissue through the sympathetic nervous system. In addition, knockdown of NPY expression increased uncoupling protein 1 expression in both inguinal fat and interscapular brown adipose tissue (BAT). Consistent with the activation of BAT, DMH NPY knockdown increased energy expenditure and enhanced the thermogenic response to a cold environment. Knockdown of NPY expression also increased locomotor activity, improved glucose homeostasis, and enhanced insulin sensitivity. Together, these results demonstrate critical roles of DMH NPY in body weight regulation through affecting food intake, body adiposity, thermogenesis, energy expenditure, and physical activity.

Accordingly, the present invention relates to the field of obesity. More specifically, the present invention provides methods useful in treating obesity and obesity-associated conditions. In a specific embodiment, a method for treating obesity comprises administering an effective amount of an agent that inhibits expression of neuropeptide Y (NPY). In a more specific embodiment, the agent inhibits expression of NPY in the dorsomedial hypothalamus (DMH). The agent can be selected from the group consisting of a small molecule, a protein, a polypeptide, an RNA interference agent, an antibody, an antisense oligonucleotide, and an enzymatic nucleic acid. In one embodiment, the RNA interference agent is siRNA. In another embodiment, the RNA interference agent is shRNA.

The present invention also provides compositions useful in treating obesity and obesity-associated conditions. In particular embodiments, a recombinant nucleic acid construct comprises a nucleic acid sequence encoding an oligonucleic acid, wherein the oligonucleic acid comprises at least one sequence substantially complementary to at least a part of the neuropeptide Y (NPY) gene or transcript thereof, and wherein the oligonucleic acid inhibits or reduces the expression of NPY. The oligonucleic acid can be selected from the group consisting of an antisense oligonucleotide, an RNA interference (RNAi) agent, and an enzymatic nucleic acid. In a specific embodiment, the oligonucleic acid is an RNAi agent. In a more specific embodiment, the RNAi agent is a short hairpin RNA. In another embodiment, the construct further comprises a U6 promoter operably linked to the nucleic encoding the oligonucleic acid. In yet another embodiment, the oligonucleic acid is substantially complementary to base pairs 257-277 of SEQ ID NO:1. In yet another embodiment, the oligonucleic acid is an RNAi agent comprising at least one sequence that is fully complementary to a target sequence of about 20 to about 30 nucleotides of the NPY transcript.

In another embodiment, the construct further comprises at least one dorsomedial hypothalamus (DMH)-specific transcription regulating sequence operably linked to the at least one nucleic acid sequence encoding an oligonucleic acid. The hypothalamus-specific transcription regulating sequence can be selected from the group consisting of DMH-specific promoters, DMH specific enhancers, and transcription regulating sequences that induce expression in cells of the DMH.

The present invention also provides vectors comprising the construct described herein. In a specific embodiment, the vector is a viral-based vector. In a more specific embodiment, the viral-based vector is an adenovirus-associated viral-based vector. In another embodiment, a host cell comprises a vector of the present invention. In yet another embodiment, the present invention provides pharmaceutical compositions comprising the construct described herein.

The present invention further provides methods for treating an obesity-associated condition or symptom associated therewith in a subject in need thereof comprising administering to the subject an effective amount of an agent that inhibits expression of neuropeptide Y (NPY). The obesity-associated condition can be selected from the group consisting of cardiovascular disease, type 2 diabetes, cancer, steatohepatitis, and osteoarthritis. In another embodiment, a method for reducing fat cell mass in a subject in need thereof comprises administering to the subject an effective amount of an agent that inhibits expression of neuropeptide Y (NPY).

In an alternative embodiment, a method for treating obesity in a subject comprises administering an agent that transforms white adipose tissue into brown adipose tissue in the subject thereby treating obesity. The agent may comprise an agent that inhibits NPY expression. More specifically, the agent inhibits NPY expression in the DMH. According to the methods described herein, the agent comprises a recombinant nucleic acid construct comprising a nucleic acid sequence encoding an oligonucleic acid comprising at least one sequence substantially complementary to at least a part of the neuropeptide Y (NPY) gene or transcript thereof, and wherein the oligonucleic acid inhibits expression of NPY. In particular embodiments, the recombinant nucleic acid construct is administered locally to specific fat depots or is administered systemically:

### BRIEF DESCRIPTION OF THE FIGURES

FIG. 1 displays the results of Adeno-associated virus-mediated knockdown of NPY expression in the dorsomedial hypothalamus. FIG. 1A shows the bilateral injections of adeno-associated virus (AAV) vectors into the dorsomedial hypothalamus (DMH). FIG. 1B is a representative micrograph showing humanized Renilla green fluorescent protein (hrGFP) expression in the DMH post-viral DMH injection as examined under fluorescence microscopy. In FIG. 1C, ³⁵S-labeled in situ hybridization histochemistry shows suppressed Npy expression in the DMH (pseudored) in rats receiving bilateral DMH injections of AAVshNPY compared with rats receiving bilateral DMH injections of AAVshCTL. In FIGS. 1D and 1E, mean±SEM Npy mRNA levels significantly decreased in the DMH of AAVshNPY rats at 1, 2, and 4 weeks after viral injection compared with AAVshCTL rats (FIG. 1D), but Npy mRNA levels in the arcuate nucleus (ARC) did not differ between AAVshCTL and AAVshNPY rats at any time points (FIG. 1E). n=5 per group. *p<0.05 compared with AAVshCTL rats. f: fornix; 3v: the third ventricle.

FIG. 2 shows the effects of DMH NPY knockdown on food intake, body weight, and glucose tolerance. FIG. 2A shows body weight gain in AAVshCTL and AAVshNPY rats with access to a regular chow (RC) or high-fat (HF) diet. OGTT, time of oral glucose tolerance test. FIG. 2B shows daily food intake in the four groups of rats. FIG. 2C displays blood glucose response to oral glucose administration. AUC indicates the area under the curve. FIG. 2D present the plasma insulin response to oral glucose administration. Values are means±SEM n=6 rats per group. *p<0.05 compared with AAVshCTL RC rats, ^{#}p<0.05 compared with AAVshNPY RC rats and ^{§}p<0.05 compared with AAVshCTL HF rats.

FIG. 3 demonstrates that DMH NPY knockdown promotes development of brown adipocytes in inguinal WAT. FIG. 3A shows fat weights at three different sites in the four groups of rats. In FIG. 3B, the color of inguinal WAT became dark (brownish) in AAVshNPY rats compared to that of AAVshCTL rats as indicated by black arrows. FIG. 3C is a representative hematoxylin and eosin (H&E) stain showing unilocular adipocytes in inguinal WAT of AAVshCTL rats (upper left) and multilocular adipocytes (brown-like adipocytes) in inguinal WAT of AAVshNPY rats (upper middle). Mitochondrial uncoupling protein 1 (UCP1) was detected in inguinal adipocytes in AAVshNPY rats (green, lower middle) by using immunostaining with anti-UCP1 antibody, and nuclei (blue) were counterstained by DAP1 (40,6-Diamidino-2-phenylindole). UCP1 immunoreactive (green) unilocular adipocytes were also detected in inguinal WAT of AAVshNPY rats (lower right), but were undetectable in inguinal WAT of AAVshCTL rats (lower left). Scale bar indicates 20 μm. FIG. 3D shows that Ucp1 mRNA was expressed in the inguinal fat of AAVshNPY rats as determined by RT-PCR. FIG. 3E shows that UCP1 protein was produced in the inguinal fat of AAVshNPY rats as determined by western blot. FIG. 3F shows mRNA expression levels in the inguinal adipose tissue including Ucp1, peroxisome proliferator-activated receptor-γ (PPAR-γ)-coactivator-1α (Pgc-1α), Ppar-γ, fatty-acid synthesis (Fas), and carnitine palmitoyltransferase 1α (Cpt 1α). FIG. 3G shows Ucp1 mRNA expression in the interscapular brown adipose tissue. Values are means±SEM (n=6) rats per group. *p<0.05 compared with AAVshCTL RC rats, ^{#}p<0.05 compared with AAVshNPY RC rats, and ^{§}p<0.05 compared with AAVshCTL HF rats.

FIG. 4 shows sympathetic denervation in the inguinal adipose tissue of AAVshNPY rats. FIG. 4A displays the schedule of sympathetic denervation experiment. FIG. 4B demonstrates that the development of brown adipocytes in the inguinal WAT was prevented by the local injection of 6-hydroxydopamine (6-OHDA) compared to the contralateral injection of saline in AAVshNPY rats. FIG. 4C shows NE concentration in inguinal fat pads. FIG. 4D presents H&E stains showing clusters of multilocular adipocytes (brownlike adipocytes) in the side of saline-treated inguinal fat pad, but barely in the 6-OHDA-treated side. Scale bar indicates 50 μm. In FIG. 4E, UCP1 immunostaining was highly detected in the side of saline treated inguinal fat pad, but not in the 6-OHDA-treated side. As shown in FIG. 4F, 6-OHDA treatment prevented Ucp1 mRNA expression in the inguinal adipose tissue. Values are means±SEM; n=5 rats for control group and 10 rats for NPY knockdown group. *p<0.05 compared to the saline-treated inguinal fat pad of AAVshCTL rats and ^{#}p<0.05 compared to the saline-treated inguinal fat pad of AAVshNPY rats.

FIG. 5 shows the effects of DMH NPY knockdown on locomotor activity, energy expenditure, and body-temperature response to cold environment. FIG. 5A shows locomotor activity during the 22 hr period. FIG. B displays energy expenditure during the 24 hr period. FIG. 6C, shows body temperature during the 6 hr cold exposure (6° C.). Values are means±SEM; n=7-8 rats per group. *p<0.05 compared to AAVshCTL rats.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### 1. DEFINITIONS

“Agent” refers to any and all materials that may be used as or in pharmaceutical compositions, including any and all materials such as small synthetic or naturally derived organic compounds, nucleic acids, polypeptides, antibodies, fragments, isoforms, variants, or other materials that may be used independently for such purposes, all in accordance with the present invention.

As used herein, and unless otherwise indicated, the term “complementary,” when used to describe a sequence in relation to a target sequence or target gene, means that the sequence is able to bind to the target sequence in a cellular environment in a manner sufficient to disrupt the function (e.g., replication, splicing, transcription or translation) of the gene comprising the target sequence. The binding may result from interactions such as, but not limited to, nucleotide base parings (e.g., A-T/G-C). In particular embodiments of the invention, a sequence is complementary when it hybridizes to its target sequence under high stringency, e.g., conditions for hybridization and washing under which nucleotide sequences, which are at least 60 percent (preferably greater than about 70, 80, or 90 percent) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which is incorporated herein by reference. Another example of stringent hybiridization conditions is hybridization of the nucleotide sequences in 6× sodium chloride/sodium citrate (SSC) at about 45° C., followed by 0.2×SSC, 0.1% SDS at 50-65° C. Particularly preferred stringency conditions are hybridization in 6× sodium chloride/sodium citrate (SSC) at about 45° C., followed by one or more washes in 0.2.xSSC, 0.1% SDS at 50° C. Depending on the conditions under which binding sufficient to disrupt the functions of a gene occurs, a sequence complementary to a target sequence within the gene need not be 100 percent identical to the target sequence. For example, a sequence can be complementary to its target sequence when at least about 70, 80, 90, or 95 percent of its nucleotides bind via matched base pairings with nucleotides of the target sequence.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and. RNA can be synthesized naturally (e.g, by DNA replication or transcription of DNA or RNA, respectively). RNA can be post-transcriptionally modified. DNA and RNA can also be chemically synthesized. The terms “target mRNA” and “target transcript” are synonymous as used herein.

As used herein, and unless otherwise indicated, the terms “inhibiting” or “inhibiting the synthesis or expression” of a gene (e.g., NPY) means impeding, slowing or preventing one or more steps by which the end-product protein encoded by the gene is synthesized. Typically, the inhibition involves blocking of one or more steps in the gene's replication, transcription, splicing or translation, through a mechanism that comprises recognition of a target site located within the gene or transcript sequence based on sequence complementation. In a specific embodiment, inhibition of NPY reduces the amount of NPY protein in the cell by greater than about 20%, 40%, 60%, 80%, 85%, 90%, 95%, or 100%. The amount of NPY protein can be determined by well-known methods including, but are not limited to, densitometer, fluorometer, radiography, luminometer, antibody-based methods and activity measurements.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g, by DNA replication or transcription of DNA or RNA, respectively). RNA can be post-transcriptionally modified. DNA and RNA can also be chemically synthesized. The terms “target mRNA” and “target transcript” are synonymous as used herein.

The term “RNA interference” (“RNAi”) refers to selective intracellular degradation of RNA (also referred to as gene silencing). RNAi also includes translational repression by microRNAs or siRNAs acting like microRNAs. RNAi can be initiated by introduction of small interfering RNAs (siRNAs) or production of siRNAs intracellularly (e.g., from a plasmid or transgene), to silence the expression of one or more target genes. Alternatively, RNAi occurs in cells naturally to remove foreign RNAs (e.g., viral RNAs). Natural RNAi proceeds via dicer-directed fragmentation of precursor dsRNA which direct the degradation mechanism to other cognate RNA sequences.

The term “small interfering RNA” (“siRNA”), also referred to in the art as “short interfering RNAs,” refers to an RNA (or RNA analog) comprising between about 10-60 nucleotides (or nucleotide analogs) that is capable of directing or mediating RNA interference. The term “siRNA” includes both double stranded siRNA and single stranded siRNA. Generally, as used herein the term “siRNA” refers to double stranded siRNA (as compared to single stranded or antisense RNA). The term “short hairpin RNA” (“shRNA”) refers to an siRNA (or siRNA analog) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

The term “RNAi agent” or “RNAi-inducing entity” refers to an RNA species (other than a naturally occurring molecule not modified by the hand of man or transported into its location by the hand of man) whose presence within a cell results in RNAi and leads to reduced expression of an RNA to which the RNAi agent is targeted. The RNAi agent may be, for example, an siRNA or an shRNA. In certain embodiments, an siRNA may contain a strand that inhibits expression of a target RNA via a translational repression pathway utilized by endogenous small RNAs referred to as microRNAs. In certain embodiments, an shRNA may be processed intracellularly to generate an siRNA that inhibits expression of a target RNA via this microRNA translational repression pathway. Any “target RNA” may be referred to as a “target transcript” regardless of whether the target RNA is a messenger RNA. The terms “target RNA” and “target transcript” are used interchangeably herein. The term RNAi agent or RNAi-inducing agent encompasses RNAi agents and vectors (other than naturally occurring molecules not modified by the hand of man as described above) whose presence within a cell results in RNAi and leads to reduced expression of a transcript to which the RNAi agent is targeted. At the level of post-transcriptional control, entirely new mechanisms of gene regulation have been discovered, typified by a large and growing class of ˜22-nucleotide-long non-coding RNAs, known as microRNAs (miRNAs), which function as repressors in all known genomes. The term “RNAi agent” or “RNAi-inducing entity” also encompasses such miRNAs.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

When used to describe the sequences of siRNAs, for example, the term “corresponding to,” as used herein, means that an siRNA has a sequence that is identical or complementary to the portion of target mRNA that is transcribed from the denoted DNA sequence.

As used herein, and unless otherwise indicated, the term “antisense oligonucleotide” refers to an oligonucleotide having a sequence complementary to a target DNA or RNA sequence.

As used herein, the term “antisense strand” of an siRNA or RNAi agent e.g., an antisense strand of an siRNA duplex or siRNA sequence, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” or “second strand” of a siRNA or RNAi agent e.g., an antisense strand of an siRNA duplex or siRNA sequence, refers to a strand that is complementary to the antisense strand or first strand. Antisense and sense strands can also be referred to as first or second strands, the first or second strand having complementarity to the target sequence and the respective second or first strand having complementarity to said first or second strand.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

A “target gene” is a gene whose expression is to be selectively inhibited or “silenced”, e.g., NPY. In certain embodiments, this silencing is achieved by cleaving the mRNA of the target gene by a siRNA/miRNA that is created from an engineered RNA precursor by a cell's RNAi system or non-coding RNAs. One portion or segment of a duplex stem of the RNA precursor is an anti-sense strand that is complementary, e.g., fully complementary, to a section of about 18 to about 40 or more nucleotides of the mRNA of the target gene.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., an NPY inhibitor or an NPY agonist. The effect may be prophylactic in terms of completely or partially preventing a particular outcome, disease or symptom thereof and/or may be therapeutic in terms of a partial or complete cure for a disease and/or adverse affect attributable to the disease. “Treatment,” as used herein, covers any treatment of a disease or condition in a subject, particularly in a human, and includes: (a) preventing the disease or condition from occurring in a subject which may be predisposed to the disease or condition but has not yet been diagnosed as having it; (b) inhibiting the disease or condition, i.e., arresting its development; and (c) relieving the disease or condition, e.g., causing regression of the disease or condition, e.g., to completely or partially remove symptoms of the disease or condition. In particular embodiments, the term is used in the context of treating a subject with a pulmonary condition.

“Administering” includes routes of administration which allow the compositions of the present invention to perform their intended function, e.g., treating obesity or obesity-associated conditions. A variety of routes of administration are possible including, but not limited to, parenteral (e.g., intravenous, intraarterial, intramuscular, subcutaneous injection), oral (e.g., dietary), inhalation (e.g., aerosol to lung), topical, nasal, rectal, or via slow releasing microcarriers depending on the disease or condition to be treated. In particular embodiments, the route of administration is parenteral. Formulation of the compound to be administered will vary according to the route of administration selected (e.g., solution, emulsion, gels, aerosols, capsule). An appropriate composition can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. See generally, Remington's Pharmaceutical Science, 16th Edition, Mack, Ed. (1980)).

An “effective amount” includes those amounts of the composition of the present invention which allow it to perform its intended function, e.g., treating or preventing, partially or totally, obesity as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 100 mg/kg/day, from about 0.1 mg/kg/day to about 10 mg/kg/day. For example, an siRNA is delivered to a subject in need thereof at a dosage of from about 0.1 mg/kg/day to about 5 mg/kg/day. A therapeutically effective amount of a composition of the present invention can be administered by an appropriate route in a single dose or multiple doses. Further, the dosages of the composition can be proportionally increased or decreased as indicated by the exigencies of the therapeutic or prophylactic situation.

### III. NPY INHIBITORS

In one aspect, the present invention comprises the administration of an effective amount of an NPY inhibitor. The term “NPY inhibitor” refers to an agent, as defined herein, that inhibits the synthesis of NPY. An NPY inhibitor may comprise, but is not limited to, a small molecule, a protein, a polypeptide, an RNA interference agent, an antibody, an antisense oligonucleotide and an enzymatic nucleic acid. In particular embodiments, the RNA interference agent can be a small interfering RNA, short hairpin RNA or a microRNA.

Accordingly, in certain aspects of the present invention, the expression of NPY may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase Ill promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-20 (2002).

In particular embodiments, the present invention features “small interfering RNA molecules” (“siRNA molecules” or “siRNA”) and “microRNAs” (miRNAs), methods of making siRNA and miRNA molecules and methods for using siRNA and miRNA molecules (e.g., research and/or therapeutic methods). The siRNAs and miRNAs of the present invention encompass any siRNAs and miRNAs that can modulate the selective degradation of NPY. General methods for making such molecules are described herein and are known to those of ordinary skill in the art.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to a target NPY mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA molecules of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNAs of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoester group connecting to adjacent ribonucleotides may be replaced by a modified group, e.g., a phosphothioate group. In sugar-modified ribonucleotides, the 2′ OH-group may be replaced by a group selected from H, OR, R, halo, SH, SR, NH₂, NHR, NR₂ or ON, wherein R is C₁-C₆ alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Derivatives of siRNAs may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNAs of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PROC. NATL. ACAD. SCI. USA 2264-68 (1990), and as modified as in Karlin and Altschul 90 PROC. NATL. ACAD. SCI. USA 5873-77 (1993). Such an algorithm is incorporated into the BLAST programs (version 2.0) of Altschul, et al., 215 J. MOL. BIOL. 403-10 (1990).

In another embodiment, the alignment may optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) NUCLEIC ACIDS RES. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional hybridization conditions include, but are not limited to, hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length can be about 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na⁺])+0.41(% G+C)−(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the NPY mRNA.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

The terms “enzymatic nucleic acid molecule” or “enzymatic oligonucleic acid” refers to a nucleic acid molecule which has complementarity in a substrate binding region to a specified gene target, and also has an enzymatic activity which is active to specifically cleave target RNA, thereby silencing the target gene. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme.

The present invention also provides ribozymes as a tool to inhibit NPY expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well known catalytic sequence responsible for mRNA cleavage. See U.S. Pat. No. 5,093,246. Using the known sequence of the target NPY mRNA, a restriction enzyme-like ribozyme can be prepared using standard techniques.

The possibility of designing ribozymes to cleave any specific target RNA has rendered them valuable tools in both basic research and therapeutic applications. In therapeutics, ribozymes have been exploited to target viral RNAs in infectious diseases, dominant oncogenes in cancers, and specific somatic mutations in genetic disorders Ribozymes and ribozyme analogs are described, for example, in U.S. Pat. No. 5,631,115, No. 5,545,729 and No. 5,436,330. Because ribozymes, unlike antisense molecules, are catalytic, a lower intracellular concentration is required for efficiency.

While ribozymes that cleave mRNA at site-specific recognition sequences can be used to destroy particular mRNAs, other ribozymes include hammerhead ribozymes. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art, see for example WO 2004/041197.

Ribozymes also include RNA endoribonucleases (hereinafter “Cech-type ribozymes”) such as the one which occurs naturally in 25 Tetrahymena thermophila (known as the IVS, or L-19 IVS RNA) and which has been extensively described by Thomas Cech and collaborators. See, e.g., WO 1988/004300). The Cech-type ribozymes have an eight base pair active site that hybridizes to a target RNA sequence thereafter cleavage of the target RNA takes place. The present invention encompasses those Cech-type ribozymes that target eight base-pair active site sequences.

Another agent capable of silencing a target gene is a DNAzyme molecule, which is capable of specifically cleaving an mRNA transcript or a DNA sequence of a target gene. DNAzymes are single-stranded polynucleotides that are capable of cleaving both single- and double-stranded target sequences. Examples of construction and amplification of synthetic, engineered DNAzymes recognizing single- and double-stranded target cleavage sites are disclosed in U.S. Pat. No. 6,326,174.

There are currently two basic types of DNAzymes, and both of these were identified by Santoro and Joyce (see, e.g., U.S. Pat. No. 6,110,462). The 10-23 DNA enzyme comprises a loop structure that connects two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms.

The expression of an NPY gene can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of NPY may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target NPY DNA or mRNA can also be used in connection with this invention without departing from the spirit of the invention. In a specific embodiment, using techniques known to those of ordinary skill in the art, the present invention contemplates affecting the promoter region of an NPY gene to effectively switch off transcription.

Accordingly, compositions of the present invention including, for example, RNAi inducing agents, antisense molecules, and the like, can be designed using the known NPY mRNA sequence (humans, GenBank NM000905) (SEQ ID NO:1). In certain embodiments, the target sequence (e.g., for siRNA) is in the region of base pairs 257-277 of human NPY mRNA (AACCTCATCACCAGGCAGAGA) (SEQ ID NO:2), wherein the two underline bases are mismatched base pairs compared to the rat sequence. Using methods known in the art, a skilled artisan can design and test further compositions useful for inhibiting NPY expression.

In rats, the shRNA sequence (double strands) for generating a recombinant shRNA expression vector using the U6 promoter comprises: 5′-TCTCATCACCAGACAGAGATTCAAGAGATCTCTGTCTGGTGATGAGATTTT TT-3′ (SEQ ID NO:3) 5′-AATTAAAAAATCTCATCACCAGACAGAGATCTCTTGAATCTCTGTCTGGTGAT GAGAGGCC-3′ (SEQ ID NO:4). The siRNA target sequence, AATCTCATCACCAGACAGAGA (SEQ ID NO:5), in the region of base pairs 240-260 based on the rat NPY mRNA sequence (GenBank NM_{—}012614) (SEQ ID NO:6) was used.

One or more of the following guidelines may be used in designing the sequence of siRNA and other nucleic acids designed to bind to a target NPY mRNA, e.g., shRNA, stRNA, antisense oligonucleotides, ribozymes, and the like, that are advantageously used in accordance with the present invention.

Beginning with the AUG start codon of an NPY gene, each AA dinucleotide sequence and the 3′ adjacent 16 or more nucleotides are potential siRNA targets. In a specific embodiment, the siRNA is specific for a target region that differs by at least one base pair between the wild type and mutant allele or between splice variants. In dsRNAi, the first strand is complementary to this sequence, and the other strand identical or substantially identical to the first strand. siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules may have a 3′ overhang of 2 nucleotides, such as TT. The overhanging nucleotides may be either RNA or DNA. In one embodiment, it may be desirable to choose a target region wherein the mismatch is a purine:purine mismatch.

Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website (http://www.ncbi.nih.gov). Select one or more sequences that meet the criteria for evaluation.

Another method includes selecting in the sequence of the target mRNA, a region located from about 50 to about 100 nt 3′ from the start codon. In this region, search for the following sequences: AA(N19)TT or AA(N21), where N=any nucleotide. The GC content of the selected sequence should be from about 30% to about 70%, preferably about 50%. To maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of a siRNA, sbRNA or stRNA (as well as other antisense oligonucleotides) to the mRNA. Further general information about the design and use of siRNA may be found in “The siRNA User Guide,” available at The Max-Planck-Institut fur Biophysikalishe Chemie website (http://www.mpibpc.mpg.de).

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome.

Delivery of the compositions of the present invention (e.g., siRNAs, antisense oligonucleotides, or other compositions described herein) into a patient can either be direct, e.g., the patient is directly exposed to the compositions of the present invention or compound-carrying vector, or indirect, e.g., cells are first transformed with the compositions of this invention in vitro, then transplanted into the patient for cell replacement therapy. These two approaches are known as in vivo and ex vivo therapy, respectively.

In the case of in vivo therapy, the compositions of the present invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector, by direct injection of naked DNA, by coating with lipids or cell-surface receptors or transfecting agents, encapsulation in liposomes, nanoparticles, microparticles, or microcapsules, by administering them in linkage to a peptide which is known to enter the cell or nucleus, or by administering them in linkage to a ligand subject to receptor-mediated endocytosis which can be used to target cell types specifically expressing the receptors. Further, the compositions of the present invention can be targeted in vivo for cell specific uptake and expression, by targeting a specific receptor. See, e.g., WO93/14188, WO 93/20221, WO 92/22635, WO92/20316, and WO 92/06180.

Ex vivo therapy involves transferring the compositions of the present invention to cells in tissue culture by methods well-known in the art such as electroporation, transfection, lipofection, microinjection, calcium phosphate mediated transfection, nanosystems, cell fusion, chromosome-mediated gene transfer, microcell-mediated gene transfer, spheroplast fusion, and infection with a viral vector containing the nucleic acid sequences. These techniques should provide for the stable transfer of the compositions of this invention to the cell, so that they are expressible by the cell and preferably heritable and expressible by its cell progeny. In particular embodiments, the method of transfer includes the transfer of a selectable marker to the cells. The cells are then placed under selection to isolate those cells that have taken up and are expressing the transferred compositions. The resulting recombinant cells can be delivered to a patient by various methods known in the art. Examples of the delivery methods include, but are not limited to, subcutaneous injection, skin graft, and intravenous injection.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Animals.

Male Sprague-Dawley rats were purchased from Charles River Laboratories, Inc., and were individually housed on a 12:12 hr light-dark cycle (lights on at 0600 hr) in a temperature-controlled colony room (22° C.-24° C.) with ad libitum access to tap water and standard laboratory rodent chow, except where noted. All procedures were approved by the Institutional Animal Care and Use Committee at the Johns Hopkins University.

AAV-Mediated RNAi Vector.

As described previously (Yang et al., 2009), the plasmids (pAAVshNPY or pAAVshCTL) containing the two cassettes of CMV (cytomegalovirus) promoter-driven hrGFP marker and mouse U6 promoter-driven shRNA (shNPY or shCTL), flanked by AAV2 inverted terminal repeats (ITR), were constructed using the pAAV-hrGFP plasmid (Stratagene). AAV-293 cells (Stratagene) cultured in DMEM growth medium (containing 4.5 g/L glucose, 110 mg/L sodium pyruvate, and 4 mM L-glutamine; Invitrogen, Carlsbad, Calif.) supplemented with 10% (v/v) heat-inactivated fetal bovine serum were used for viral packaging. Three plasmids of pAAVshNPY (or pAAVshCTL), pHelper (carrying adenovirus-derived genes) and pAAV-RC (carrying AAV-2 replication and capsid genes) were co-transfected into AAV-293 cells according to the manufacturer's protocol (Stratagene). Three days after transfection, cells were harvested, and the recombinant viral vector AAVshNPY (or AAVshCTL) was purified using the AAV purification kit (Virapur, LLC) and concentrated using Centricon YM-100 (Millipore) according to the manufacturers' protocols. Virus titers were determined using quantitative PCR and ˜1×10⁹ particles/site were used for each virus injection.

### AAV-Mediated Knockdown of NPY Expression in the DMH.

For determining the effects of the vector AAVshNPY on Npy gene expression in the DMH, 15 rats weighing 270-300 g received bilateral DMH injections of AAVshNPY and were euthanized (n=5) at 1, 2, and 4 weeks postviral injection. Five control rats received control vector AAVshCTL injections and were euthanized at 4 weeks post-viral injection. DMH viral injection was made as previously described (Yang et al., 2009). Briefly, 0.5 μl/site (˜1×10⁹ particles/site) of recombinant AAV vectors were injected into the DMH with coordinates: 3.1 mm caudal to bregma, 0.4 mm lateral to midline, and 8.6 mm ventral to skull surface at a rate of 0.1 μl/min for 5 min-and the injector remained in place for additional 5 min before removal. After euthanization, coronal sections (14 μm) through the hypothalamus were prepared, and the sections containing hrGFP expression were examined on a Zeiss Axio Imager (Carl Zeiss Microlmaging, Inc.). Levels of Npy mRNA expression at areas of the DMH and the ARC (3.0-3.5 mm posterior to bregma [Paxinos and Watson, 2005]) were examined using in situ hybridization with ³⁵S-labeled antisense riboprobes of NPY as previously described (Yang et al., 2009).

Effects of DMH NPY Knockdown on Food Intake and Body Weight.

Following determination of the effects of AAVshNPY on NPY expression, 24 rats weighing 130-150 g were randomly assigned to either bilateral DMH injections of AAVshNPY or AAVshCTL (n=12/group) as described above with coordinates: 2.3 mm caudal to bregma, 0.4 mm lateral to midline, and 7.6 mm ventral to skull surface. Rats had ad libitum access to regular chow (15.8% fat, 65.6% carbohydrate, and 18.6% protein in kcal %; 3.37 kcal/g; PMI Nutrition International, LLC). Five weeks postviral injection, half the rats from each group were switched to ad libitum access to a high-fat diet (60% fat, 20% carbohydrate, and 20% protein in kcal %; 5.2 kcal/g; Research Diets; New Brunswick, N.J.). Food intake was measured weekly and body weight was determined daily. Glucose tolerance tests were conducted 12 weeks post-viral injection. At 16 weeks post-viral injection, rats were euthanized and the adipose tissues were collected and analyzed.

Glucose Tolerance Test.

Following an overnight fast, rats were administered oral glucose (2 g/kg) by gavage. Tail blood was sampled before and 15, 30, 45, 60, and 120 min after giving glucose for measurements of blood glucose and plasma insulin concentrations. Blood glucose levels were determined with a FreeStyle glucometer (Abbot Diabetes Care, Inc.; Alameda, Calif.). Plasma insulin concentrations were determined by a rat insulin radioimmunoassay kit (Millipore Corporation; Billerica, Mass.).

H&E Stain and Immunostaining.

Following 4% paraformaldehyde fixation and paraffin embedding, 5 μm sections of inguinal adipose tissue were cut via a cryostat. The sections were stained with H&E, and examined on Zeiss Axio Imager. For UCP1 immunostaining, the sections were incubated with goat anti-UCP1 antibody (Santa Cruz Biotechnology, Inc.; Santa Cruz, Calif.) at 4° C. overnight. After three washes, UCP1 signals were stained with Cy2-conjugated donkey anti-goat secondary antibody (Jackson ImmunoResearch; West Grove, Pa.) at room temperature for 60 min. After final washes, the sections were counterstained with DAPI (4′6 diamidi-no-2-phenylindole, for nuclei staining), coverslipped, and examined on Zeiss Axio Imager.

Quantitative Real-Time RT-PCR.

Total RNA was extracted from each sample by using Trizol reagent (Invitrogen) and the remaining organic phase was saved for subsequent protein extraction according to the manufacturer's protocols. Two-step quantitative real time RT-PCR was performed for gene expression determination. One microgram of total RNA was reverse-transcribed into first-strand cDNA using the RevertAid First Strand cDNA Synthesis Kits (FERMENTAS, INC.; Glen Burnie, Md.), and the resulting cDNA product was then quantified using iQ SYBR Green Supermix Kit (Bio-Rad Laboratories; Hercules, Calif.) on iQ5 Multicolor Real-Time PCR Detection System (Bio-Rad Laboratories). b-actin was used as an internal control for quantification of individual mRNA. A list of primer sets included: UCP1, Forward Primer: 50-cgttccaggatccgagtcgcaga-30 (SEQ ID NO:7) and Reverse Primer: 50-tcagctcttgtcgccgggttttg-30 (SEQ ID NO:8); PGC1a, Forward Primer: 50-aatgcagcggtcttagcact-30 (SEQ ID NO:9) and Reverse Primer: 50-gtgtgaggagggtcatcgtt-30 (SEQ ID NO:10); PPAR-γ, Forward Primer: 50-gcctgcggaagccctttggtgac-30 (SEQ ID NO:11) and Reverse Primer: 50-ttggcgaacagctgggaggactc-30 (SEQ ID NO:12); FAS, Forward Primer: 50-tcgagacacatcgtttgagc-30 (SEQ ID NO:13) and Reverse Primer: 50-tcaaaaagtgcatccagcag-30 (SEQ ID NO:14); CPT1a, Forward Primer: 50-ggcagaagagatggcggtcgatg-30 (SEQ ID NO:15), and Reverse Primer: 50-ccccaagt caacggcagagcaga-30 (SEQ ID NO:16).

Western Blot.

Proteins were separated by using 4%-12% SDS-PAGE (sodium dodecyl sulfate polyacrylamide gel electrophoresis), and transferred to an ImmunBlot PVDF membrane. The membrane was then incubated with goat anti-UCP1 antibody (1:200 dilution; Santa Cruz Biotechnology), followed by incubation with horseradish peroxidase-labeled donkey anti-goat antibody (Santa Cruz Biotechnology) and detected by using Super Signal West Pico Chemiluminescent Substrate Kit (Thermo Scientific; St. Louis, Mo.).

Sympathetic Denervation.

As previously described (Rooks et al., 2005), 15 rats weighing 100-120 g received 20 microinjections of 6-hydroxydopamine (6-OHDA, 1 μl per injection, 9 mg/ml in 0.15 M NaCl containing 1% ascorbic acid [Sigma Chemical; St. Louis, Mo.]) throughout the left inguinal fat pads. Right pads received an equal volume of vehicle injections and served as within-animal controls. Two weeks after 6-OHDA injections, 10 rats received bilateral DMH injections of AAVshNPY and five rats received bilateral DMH injections of AAVshCTL as described above. Twelve weeks after 6-OHDA injections, inguinal adipose tissue was evaluated.

Norepinephrine Measurements.

Norepinephrine (NE) concentrations in the inguinal fat were determined using HPLC with electrochemical detection as previously described (Pletnikov et al., 2000; Rooks et al., 2005) with some modifications. Briefly, fat tissue was homogenized on ice by sonication in 0.1 M perchloric acid solution containing dihydroxybenzylamine as an internal standard. The extracts were centrifuged at 7000 rpm for 15 min at 4° C. and filtrated. After filtration, 15 μl of the clear homogenate were injected into the chromatographic column, the peak of NE in chromatograms of samples was identified by its retention time, and NE content was calculated and expressed as nanograms of NE per fat depot.

Locomotor Activity.

Fifteen rats weighing 130-150 g received bilateral DMH injections of AAVshNPY (n=8) or AAVshCTL (n=7) for examining locomotor activity, energy expenditure, and thermogenic response to cold environment. Four weeks post-viral injection, locomotor activity was examined in 40×40×30 cm Plexiglas test chambers with a row of infrared monitoring sensors and a Digiscan computer for data collection and analysis (Accuscan Instruments; Columbus Ohio) as previously described (Aja et al., 2006). Animals were placed into individual chambers 2 hr prior to lights out and activity was monitored in 2 hr intervals for 24 hr with access to food and water ad libitum. The first 2 hr period was considered a habituation period. Data on horizontal activity (the number of beam interruptions in 2 hr intervals) during the next 22 hr period were collected and analyzed.

Indirect Calorimetry.

Five weeks postviral injection, rats were placed into individual Oxymax chambers attached to an Oxymax Equal Flow indirect calorimetric system (Columbus Instruments; Columbus, Ohio). After 5-7 days of habituation, calorimetric oxygen consumption, and carbon dioxide production, daily body weight and food intake were measured on 3 consecutive days, and daily energy expenditure was analyzed.

Cold Exposure.

Eight weeks post-viral injection, rats were initially habituated by measurement of core body temperature using a rodent rectal probe (OAKTON Instruments; Vernon Hills, Ill.) for 3-5 days. After habituation, rats were exposed to cold environment (6° C.) for 6 hr during the light period (0900-1500 hr). Core body temperature was measured at 0, 2, 4, and 6 hr of cold exposure using a rodent rectal probe.

Statistical Analysis.

All values are presented as means±SEM. Data were analyzed by StatSoft Statistica-7 software. Data for Npy mRNA expression were analyzed using one-way ANOVA. Data for body weight and food intake were analyzed using three-way ANOVA with one repeated factor. Data for blood glucose, plasma insulin, fat mass, mRNA levels, and fat NE concentrations were analyzed using two-way ANOVA. Data for UCP1 mRNA levels from sympathetic denervation experiment, locomotor activity, and energy expenditure were analyzed using Student's t test (two-tailed). Data for body temperature were analyzed using two-way repeated-measures ANOVA. All ANOVA's were followed by pairwise multiple Fisher's LSD comparisons. p<0.05 was considered as a statistically significant difference.

### Example 1

### AAV-Mediated Knockdown of NPY Expression in the DMH

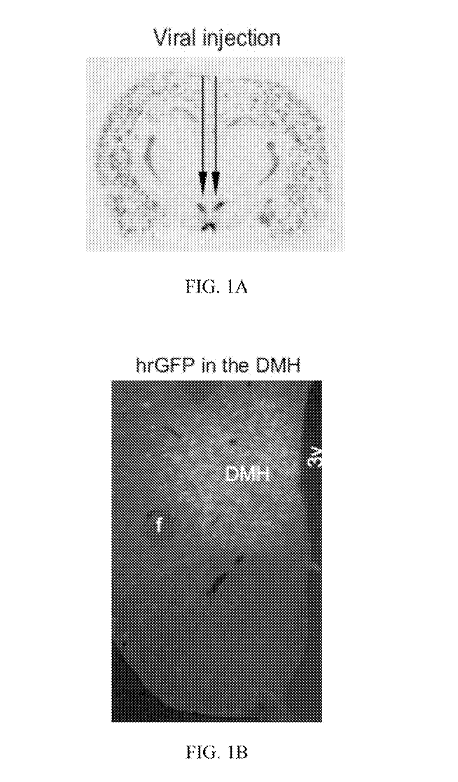

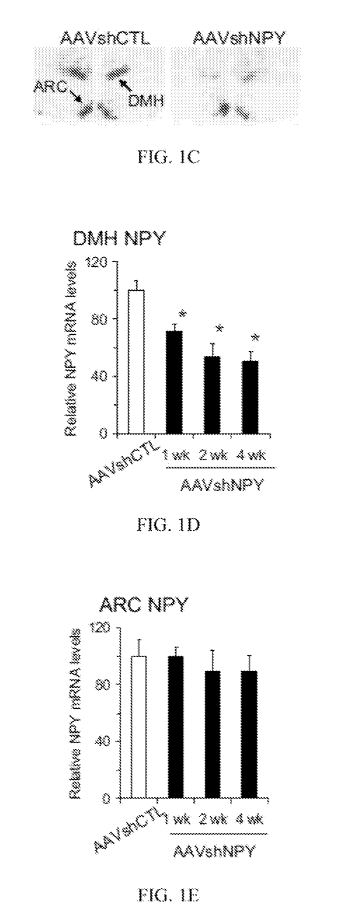

A recombinant vector of AAV-mediated RNAi was generated with NPY-specific short hairpin RNA (AAVshNPY) containing humanized Renilla green fluorescent protein (hrGFP) marker as previously reported (Yang et al., 2009). To test the idea that DMH NPY may be an important neuromodulator of energy balance under normal conditions, the effect of AAV-mediated RNAi on Npy gene expression was first determined in Sprague-Dawley rats by injecting this vector bilaterally into the DMH (FIG. 1A).

This example established that the viral vectors infected neurons within the DMH as early as 1 week after viral injection, led to a robust infection within 2 weeks (FIG. 1B, hrGFP-positive neurons), and produced significant knockdown of Npy mRNA expression in the DMH (FIG. 1C) by 28%, 47%, and 49% at 1, 2, and 4 weeks postviral injections, respectively, compared to rats receiving control vector injections (AAVshCTL, FIG. 1D). This knockdown effect was site specific since no hrGFP-positive neurons were detected in the ARC (FIG. 1B) and Npy mRNA levels were unaltered in the ARC (FIG. 1E). Consistent with the inventors' previous report (Yang et al., 2009), the effects of AAV-mediated RNAi on Npy mRNA expression in the DMH were long lasting; 16 weeks postviral injection, Npy mRNA levels remained reduced by 36% (data not shown).

### Example 2

### Effects of DMH NPY Knockdown on Regulation of Body Weight

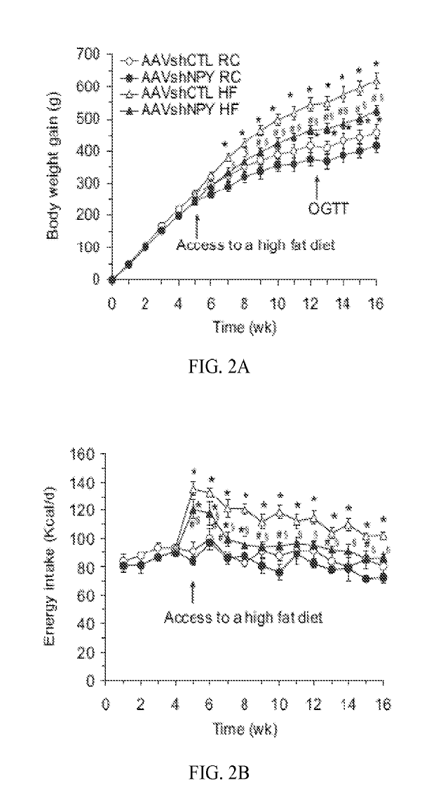

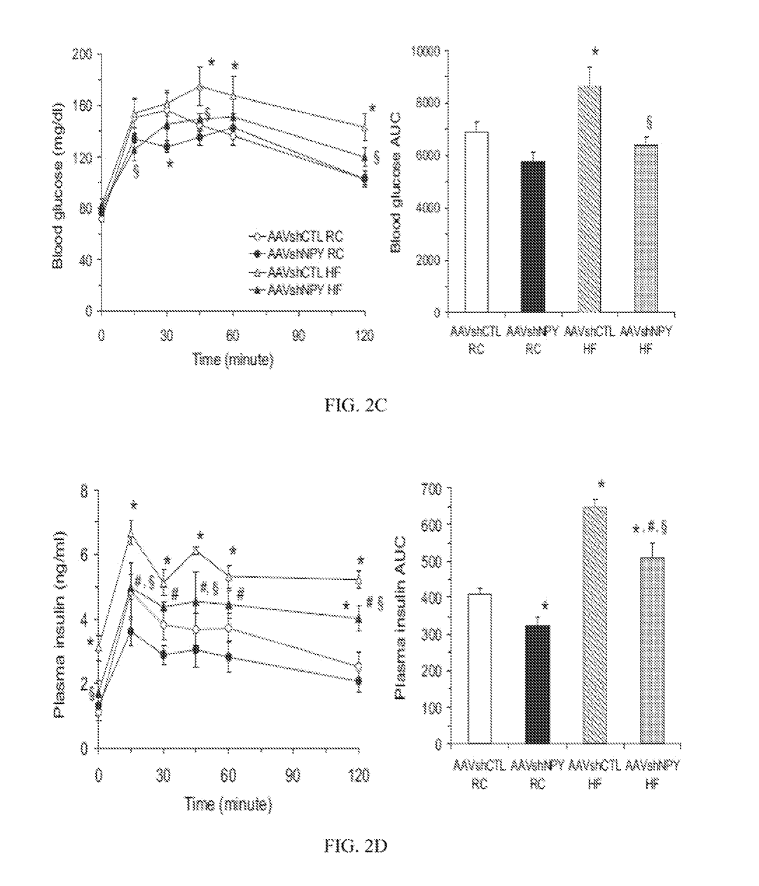

Following determination of viral-mediated knockdown of NPY expression in the DMH, whether this knockdown affects body weight regulation was examined. DMH NPY knockdown resulted in a small but significant decrease in body weight gain over the first 5 weeks post-viral injection when rats were maintained on regular chow (RC, p=0.035, FIG. 2A). The weight gain of NPY knockdown rats was reduced by about 9%. Since high-fat diet (HF) increases body weight and induces obesity, the effect of DMH NPY knockdown on HF-induced weight gain was next assessed. Half the NPY knockdown and control rats were challenged with HF at 5 weeks post-viral injection. NPY knockdown significantly reduced HF-induced increases in weight gain (p=0.023). Control rats fed HF gained significantly more weight by 2 week (p=0.026) and had gained 35% more weight by 11 weeks compared to control rats on RC. In contrast, NPY knockdown rats fed HF gained body weight more slowly, only achieving significantly increased body weight by 4 weeks (p=0.021) and having only 26% more weight by 11 weeks compared to those on RC(FIG. 2A). As a result, the body weight gain of NPY knockdown rats remained relatively normal until 7 weeks on HF compared to control rats on RC and was significantly less than control rats in 11 weeks on HF (FIG. 2A).

Because the present inventors demonstrated the effects of DMH NPY on food intake and meal patterns in both OLETF and intact rats (Yang et al., 2009), whether DMH NPY knockdown altered daily food intake was examined in the present study. Although daily energy intake did not differ between the two groups of rats over 16 weeks on RC, DMH NPY knockdown significantly reduced HF-induced hyperphagia (FIG. 2B). Both groups of rats increased daily intake dramatically upon initial access to HF, but the degree of increase and its duration were significantly reduced in NPY knockdown rats (FIG. 2B). While control rats on HF remained hyperphagic, NPY knockdown rats normalized energy intake in 4 weeks on HF (FIG. 2B).

### Example 3

### DMH NPY Knockdown Improves Glucose Homeostasis

The effects of DMH NPY knockdown on glucose homeostasis was texted next. Although oral glucose administration resulted in similar patterns of glucose clearance in NPY knockdown and control rats on RC (FIG. 2C), NPY knockdown rats required less insulin secretion to clear the glucose as indicated by a reduction in the area under the response curve of insulin in NPY knockdown rats (FIG. 2D), suggesting that downregulation of DMH NPY expression enhances insulin sensitivity. HF access caused hyperinsulinemia and impaired glucose clearance in control rats as determined by high fasting insulin levels, and elevated blood glucose and plasma insulin levels in response to oral glucose (FIGS. 2C and 2D). DMH NPY knockdown significantly ameliorated these changes. NPY knockdown rats on HF had normal glucose response to an oral glucose load (FIG. 2C) and normal fasting insulin levels (FIG. 2D) relative to control rats on RC. Although the area under the response curve of insulin in NPY knockdown rats on HF was higher than that of control rats on RC, the levels were significantly reduced compared to control rats on HF (FIG. 2D).

### Example 4

### DMH NPY Knockdown Promotes Development of Brown Adipocytes in White Adipose Tissue

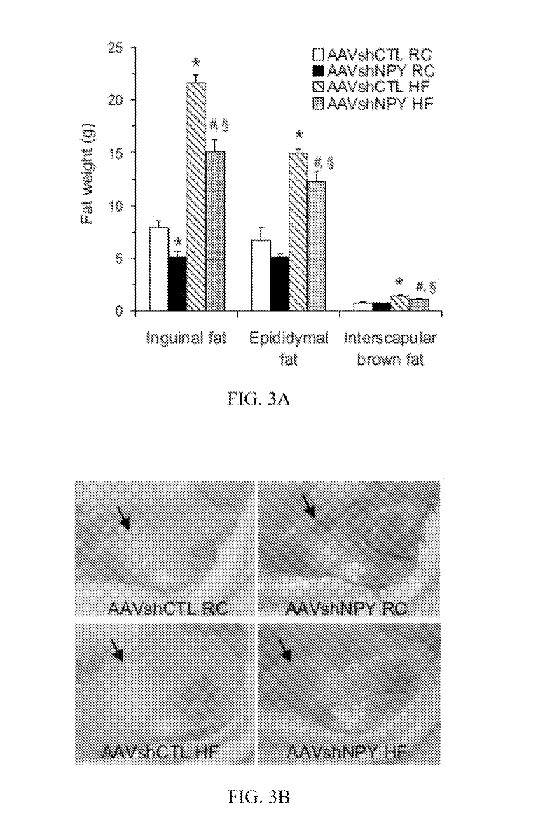

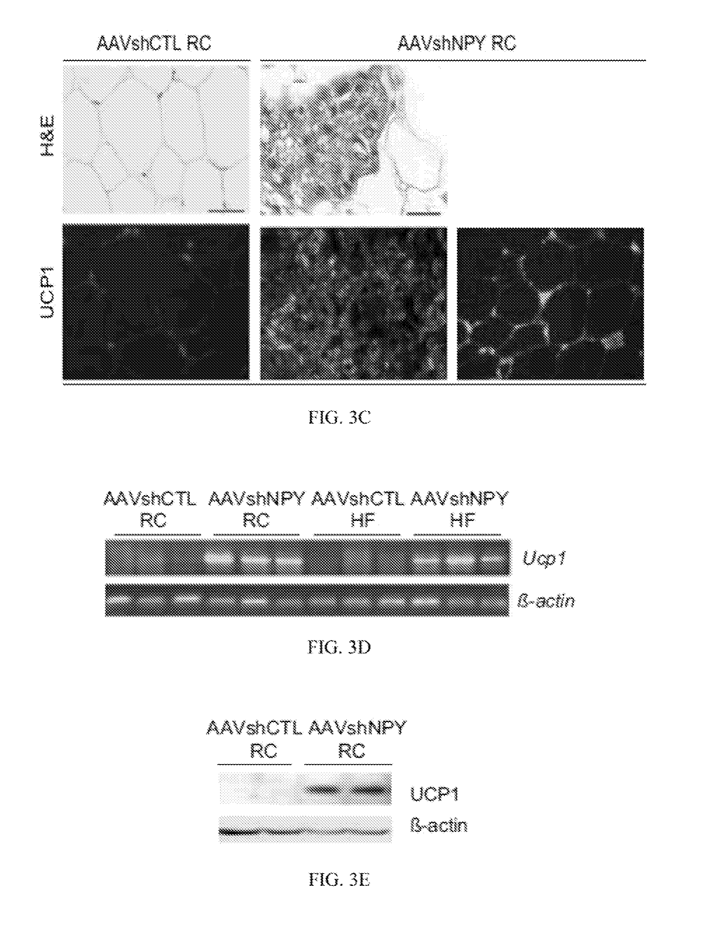

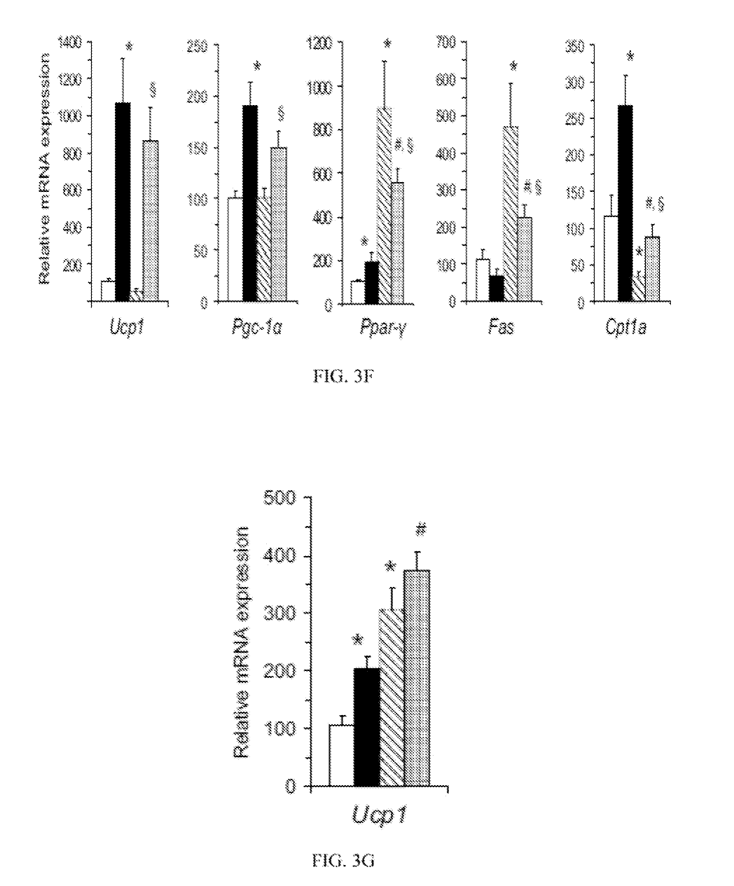

Examination of body fat mass revealed a site-specific effect of DMH NPY knockdown on adiposity. Subcutaneous inguinal fat mass was significantly decreased in NPY knockdown rats on RC compared to control rats (FIG. 3A) and the color of the inguinal fat appeared significantly darker (brownish) in NPY knockdown rats than that of control rats (FIG. 3B). This color change was also found in the subcutaneous axillary white fat areas, but not in other subcutaneous, epididymal and visceral white fat depots (including mesenteric, retroperitoneal, and perirenal fat) in NPY knockdown rats (data not shown). Moreover, while high-fat diet resulted in significant increases in fat accumulation in inguinal and epididymal white and interscapular brown fat in control rats, all these increases were significantly decreased in NPY knockdown rats on HF (FIG. 3A). Although NPY knockdown rats on HF accumulated more inguinal fat than those on RC (FIG. 3A), the fat still appeared more brown (FIG. 3B).

Inguinal adipose tissue in NPY knockdown rats was characterized next. Hematoxylin and eosin (H&E) staining revealed that inguinal adipocytes in control rats contained unilocular adipocytes, i.e., showing typical white adipocytes (FIG. 3C, upper left), whereas both the size and number of white adipocytes were reduced in inguinal adipose tissue of NPY knockdown rats. In addition, the cells formed new large clusters that contained multilocular adipocytes (brown-like adipocytes) and were surrounded by white adipocytes (FIG. 3C, upper middle). In support of brown adipocyte formation, these cells showed robust immunostaining (green) for mitochondrial uncoupling protein 1 (UCP1, a marker of brown adipose tissue, BAT; FIG. 3C, lower middle). UCP1 immunostaining (green) was also detected in a number of unilocular adipocytes in inguinal adipose tissue of NPY knockdown rats (FIG. 3C, lower right), but undetectable in those of control rats under basal conditions (FIG. 3C, lower left). Quantitative real-time RT-PCR (reverse transcriptase-polymerase chain reaction) and western blot analyses further confirmed UCP1 expression in the inguinal fat of NPY knockdown rats (FIGS. 3D and 3E). Levels of Ucp1 mRNA expression were significantly elevated in NPY knockdown rats relative to their controls (FIG. 3F). By contrast, Ucp1 mRNA expression was undetectable (or unchanged) in other white fat depots including epididymal, mesenteric, retroperitoneal, and perirenal fat in NPY knockdown rats (data not shown).

Another BAT-select gene, peroxisome proliferator-activated receptor-γ (PPAR-γ) coactivator-1α (PGC-1α) (Handschin and Spiegelman, 2006) was also examined and it was found that Pgc-1a was also highly expressed in the inguinal fat of NPY knockdown rats (FIG. 3F). Together, these results provide clear evidence that DMH NPY knockdown promotes development of brown adipocytes in inguinal white adipose tissue (WAT), or causes inguinal WAT into BAT transformation.

To test the possibility of effects of DMH NPY knockdown on adipogenesis and fat metabolism in the inguinal adipose tissue, gene expression for PPAR-γ, fatty acid synthase (FAS), and carnitine palmitoyltransferase 1a (CPT1a) was examined. PPAR-γ is an important transcription factor in the development of both white and brown fat cells (Rosen et al., 1999). Compared to control rats, Ppar-γmRNA levels were significantly increased in NPY knockdown rats, and while high-fat diet increased Ppar-γ mRNA levels in control rats, this increase was significantly reduced in NPY knockdown rats (FIG. 3F), suggesting that DMH NPY knockdown may contribute to brown adipogenesis in the inguinal fat and also limits HF-induced white fat adipogenesis. DMH NPY knockdown also affected metabolism in the inguinal fat. FAS plays a key role in fatty acid synthesis, whereas CPT1a is the rate-limiting enzyme controlling fatty acid oxidation. Compared to control rats, Cpt1a gene expression was significantly increased in the inguinal fat of NPY knockdown rats with a trend toward a decrease in Fas gene expression, indicating a shift from lipogenesis to fatty acid oxidation in this tissue (FIG. 3F). High-fat diet induced more fatty acid synthesis in control rats, with increased Fas gene expression and decreased Cpt1a gene expression, whereas DMH NPY knockdown reversed these alterations (FIG. 3F).

### Example 5

### DMH NPY Knockdown Increases BAT Activation

Brown fat is mainly deposited in the interscapular area of rats where it plays a primary role in nonshivering thermogenesis through activation of UCP1 (Cannon and Nedergaard, 2004). Whether DMH NPY knockdown affected activity of interscapular BAT was examined next. Ucp1 gene expression was significantly increased in interscapular BAT of NPY knockdown rats on RC compared to control rats (FIG. 3G), suggesting that DMH NPY knockdown results in increased BAT activity. When rats were fed HF, Ucp1 gene expression was significantly elevated in interscapular BAT in both groups (FIG. 3G), implying that HF induces thermogenesis in these two groups. Although Ucp1 gene expression was slightly higher in NPY knockdown rats than in control rats on HF, the difference was not statistically significant (FIG. 3G).

### Example 6

### Sympathetic Mediation of Development of Brown Adipocytes in WAT

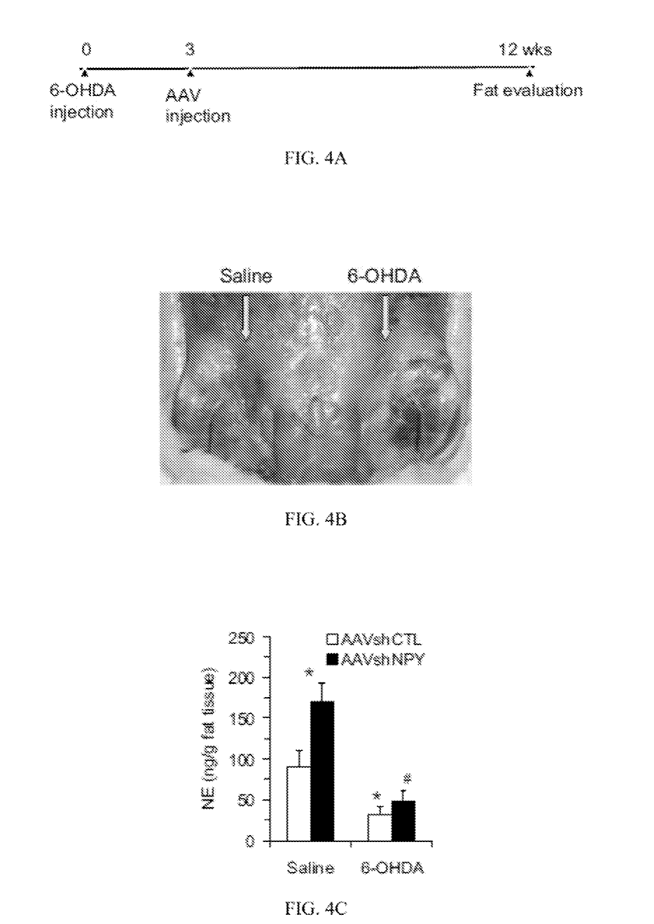

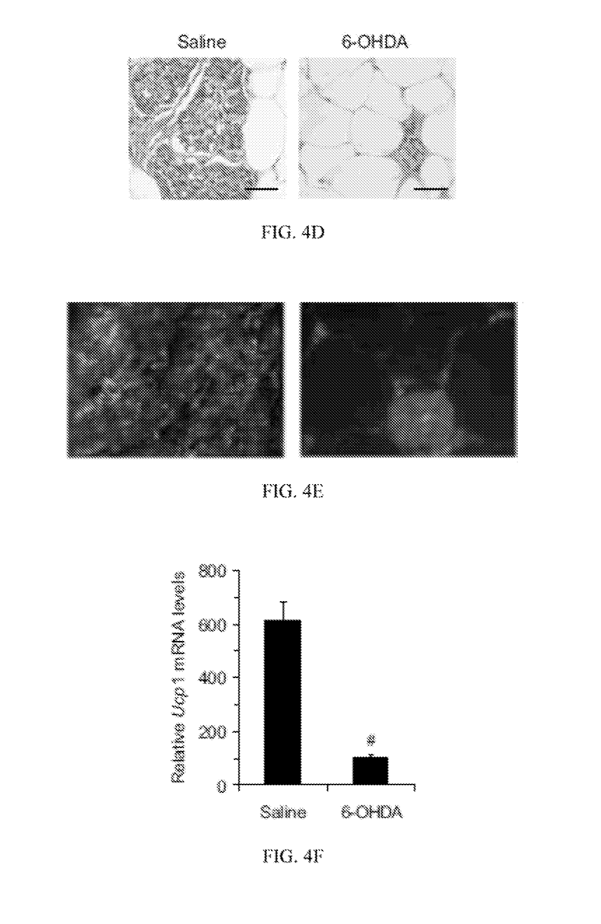

To test whether the sympathetic nervous system (SNS) mediates the development of brown adipocytes in inguinal WAT, whether sympathetic denervation altered brown adipocyte formation was examined by injecting the neurotoxin 6-hydroxydopamine (6-OHDA) unilaterally into the inguinal fat area 2 weeks prior to bilateral DMH injections of the vector AAVshNPY (FIG. 4A). At sacrifice, examination of the inguinal fat pads revealed that while the inguinal adipose tissue became dark brown on the side of saline injection, the fat tissue remained relatively white (or significantly less brown) in the side of 6-OHDA injection (FIG. 4B). DMH NPY knockdown resulted in significant increases in the level of norepinephrine (NE) within the saline-treated inguinal fat pads as compared to control rats (FIG. 4C). 6-OH DA treatment prevented this increase (FIG. 4C). Fat NE levels were significantly decreased in the side of 6-OHDA treatment relative to the control side in both groups of rats and the levels of NE within the 6-OHDA-treated inguinal fat pads did not differ between the two groups of rats (FIG. 4C). Consistent with the change of fat color, numerous clusters of brown-like adipocytes (multilocular adipocytes) were found in the side of saline-treated inguinal adipose tissue of NPY knockdown rats, whereas brown-like adipocytes were dramatically reduced by 6-OHDA treatment (FIG. 4D). Determination of UCP1 expression confirmed that 6-OHDA treatment prevented UCP1

### Example 7

### DMH NPY Knockdown Increases Energy Expenditure

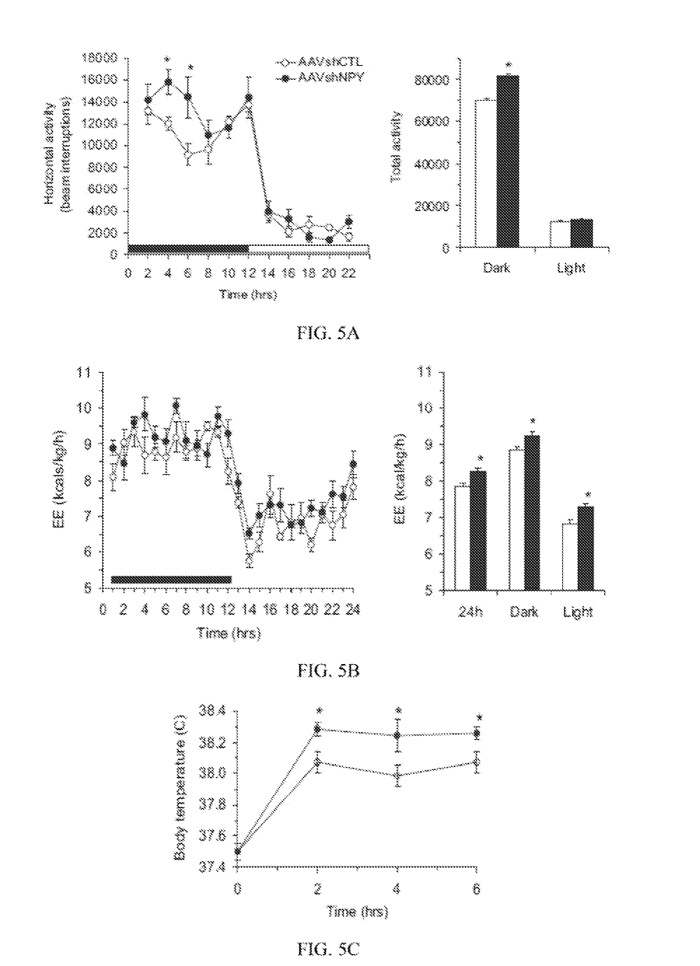

Whether DMH NPY knockdown affected energy expenditure was next examined in another cohort of NPY knockdown and control rats receiving bilateral DMH viral injections. NPY knockdown rats increased locomotor activity, particularly during the dark period (FIG. 5A). Moreover, indirect calorimetry revealed that energy expenditure was significantly increased during both dark and light phases of the circadian cycle in NPY knockdown rats (FIG. 5B). Since NPY knockdown rats showed brown adipocytes in inguinal fat and increased UCP1 expression in this inguinal and the interscapular BAT, whether DMH NPY knockdown affected thermogenesis was tested. Although core body temperature did not differ between NPY knockdown and control rats at room temperature (24° C.), NPY knockdown rats had a greater increase in thermogenic response to 6 hr of cold exposure (6° C.) compared to their control counterparts (FIG. 5C).

### Discussion

The DMH plays an important role in maintaining energy homeostasis. Lesions of the DMH resulted in hypophagia and reduced body weight (Bellinger and Bernardis, 2002). Disinhibition of neurons in the DMH provoked nonshivering thermogenesis and elevated core body temperature (Zaretskaia et al., 2002). Despite these observations, the neural mechanisms underlying these actions of the DMH remain undetermined. As described herein, a critical role for NPY in the DMH in regulating energy homeostasis was established by using AAV-mediated RNAi to knock down NPY expression in the DM H of intact rats.

The effect of DMH NPY knockdown on regulation of body weight was first assessed. DMH NPY knockdown significantly decreased diet-induced hyperphagia, resulted in slower weight gain on both RC and HF diets, and reduced body fat mass. In addition, selective effects of DMH NPY on inguinal adiposity and BAT thermogenesis were noted. DMH NPY knockdown resulted in development of brown adipocytes in inguinal WAT, increased UCP1 expression in the inguinal and interscapular BAT, and increased energy expenditure and cold induced thermogenesis. DMH NPY knockdown promoted inguinal lipid mobilization and decreased diet-induced fat accumulation. DMH NPY knockdown also resulted in increased locomotor activity. Together, these results demonstrated that DMH NPY affects multiple aspects of energy homeostasis including food intake, body adiposity, thermogenesis, energy expenditure, and physical activity.

Two types of fat, WAT and BAT, exist in mammals including in adult humans (Cypess et al., 2009; van Marken Lichtenbelt et al., 2009; Virtanen et al., 2009). While WAT stores excess calories, BAT burns fat to produce heat via nonshivering thermogenesis as a defense against cold. Both types of fat are innervated by the SNS (Bartness and Bamshad, 1998; Cannon and Nedergaard, 2004). Activation of the sympathetic innervation induces lipolysis in WAT (Fredholm and Karlsson, 1970; Weiss and Maickel, 1968) and produces thermogenesis through mitochondrial UCP1 in BAT (Cannon and Nedergaard, 2004). Sympathetic activation via treatment of β-adrenergic agonist or cold stress has also been demonstrated to cause development of brown adipocytes in white fat pads (Himms-Hagen et al., 1994; Jimenez et al., 2003; Nagase et al., 1996). In contrast, intracerebroventricular administration of NPY increases WAT lipoprotein lipase activity (suggesting increased lipid storage) and decreases BAT GDP binding activity (indicating decreased thermogenic activity) in addition to its orexigenic effect (Billington et al., 1991) and central administration of NPY also suppresses sympathetic activity in interscapular BAT in rats (Egawa et al., 1991). These observations imply that central NPY may serve as a neuromodulator of the SNS controlling both WAT lipogenesis and BAT thermogenesis. The results described herein provide support for this view and further identify DMH NPY as an important contributing factor to these effects. DMH NPY knockdown resulted in development of brown adipocytes (or white into brown adipocyte transformation) in inguinal WAT and reduced inguinal fat accumulation and that sympathetic denervation prevented this brown adipocyte formation. DMH NPY knockdown also resulted in increased UCP1 expression in the interscapular BAT. These results indicate that DMH NPY normally modulates SNS signaling to influence adiposity and energy homeostasis and that knockdown of NPY expression in the DMH results in increases in peripheral sympathetic tone selectively in the inguinal fat and interscapular brown fat. As a result, Ucp1 gene expression was upregulated in the inguinal fat and interscapular BAT of NPY knockdown rats, leading to increased thermogenesis and overall increased energy expenditure; and increases in Cpt1a gene expression with a trend for a decrease in Fas gene expression in the inguinal fat of NPY knockdown rats appear to cause increased fatty-acid oxidation in adipose tissue (increased lipid mobilization)-and overall reduce body adiposity.

Bamshad and colleagues (1998) have investigated the central nervous system origins of SNS outflow to WAT. By using viral transsynaptic retrograde tracer, they found that viral tracer was less detected in the DMH in animals receiving epididymal viral injection than those receiving inguinal injection (Bamshad et al., 1998), implying that the central nervous control of inguinal WAT is more DMH related than that of epididymal WAT. In support of this view, it was found that DMH NPY knockdown specifically affected lipid mobilization and brown adipocyte formation in inguinal WAT through the SNS. This suggests that DMH NPY is an important factor influencing sympathetic innervation in inguinal WAT, but not epididymal WAT. Overall, in combination with the evidence that the DMH is involved in thermoregulation (Dimicco and Zaretsky, 2007), the present results suggest that NPY in the DMH may serve to modulate actions of both inguinal WAT and interscapular BAT in maintaining energy homeostasis.

WAT contains mature adipocytes for storage of lipids and other types of cells including preadipocytes, fibroblasts, pericytes, endothelial cells, and various blood cells in the stromal-vascular fraction (SVF) (Ailhaud et al., 1992). Although white fat progenitor cells have been demonstrated to reside in the adipose SVF (Tang et al., 2008), types of brown fat precursor cells in WAT or whether precursor cells in the SVF of WAT possess the ability to develop into both white and brown adipocytes is unclear. Reversible physiological transdifferentiation between WAT and BAT implies that white and brown adipocytes are mixed in most fat depots in rodents (including inguinal WAT) (Cinti, 2009). Barbatelli et al. (2010) further reported that the emergence of cold-induced brown adipocytes in mouse white fat depots (including inguinal WAT) is determined predominantly by white-into-brown adipocyte transdifferentiation. This trandifferentiation is thought to be directly derived from mature white adipocytes as determined by adipocytes with intermediate features between white and brown adipocytes (referred as transdifferentiating paucilocular adipocytes) (Barbatelli et al., 2010). The present study did not find clear UCP1 immunoreactive paucilocular cells in inguinal fat of NPY knockdown rats as proposed above. In fact, numerous clusters of brownlike adipocytes surrounded by white adipocytes were found as well as various UCP1 immunoreactive unilocular adipocytes in inguinal adipose tissue of NPY knockdown rats. A significant elevation of Ppar-γ expression, an essential factor for adipogenesis, was also found in this inguinal fat tissue. Therefore, although there is still the possibility of white-into-brown adipocyte transdifferentiation in this rat model, these results imply that development of brown adipocytes in inguinal WAT resulting from DMH NPY knockdown may be directly derived from brown fat-like precursor cells in the SVF.

In addition, a role for DMH NPY in regulation of spontaneous physical activity was demonstrated. Knockdown of NPY expression in the DMH resulted in increased locomotor activity. Based on the evidence that non-exercise activity thermogenesis from spontaneous physical activity may play a pivotal role in protection against fat gain (Levine et al., 1999), these results suggest that the effect of DMH NPY on physical activity may also contribute to its influence on body weight control. Moreover, DMH NPY knockdown produced a dark phase-specific effect on locomotor activity. These results provide additional evidence indicating a potential role for DMH NPY in regulation of day-night rhythms.

The finding of a role for DMH NPY in glucose homeostasis is also intriguing. Previous studies have shown that the DMH contains both glucoreceptive and glucose-sensitive neurons and lesions of the DMH alter feeding response to exogenous glucose and insulin (Bellinger and Bernardis, 2002), implicating this region in the regulation of glucose homeostasis. As described herein, DMH NPY knockdown enhanced insulin sensitivity, improved glucose tolerance, and prevented diet-induced hyperglycemia and hyperinsulinemia. These results indicate an important role of NPY in the DMH in the regulation of glucose homeostasis. Although this effect may be a direct result of reduced NPY expression in the DMH, it may also be a consequence of the brown adipocyte formation in inguinal fat, the activation of interscapular BAT, and the resulting increased thermogenesis and the subsequent lean phenotypes. Nevertheless, alterations in DMH NPY signaling were shown to influence insulin sensitivity and glucose homeostasis, but the mechanisms through which DMH NPY acts to affect insulin action and regulate glucose levels merit further investigation.

In summary, the present inventors demonstrated the physiological importance of DMH NPY in energy homeostasis. DMH NPY affects food intake, body adiposity, thermogenesis, energy expenditure, and physical activity to regulate body weight. These results indicate that orexigenic NPY in the DMH normally serves as a key factor in maintaining energy homeostasis and also point to the DMH as a potential target site for therapies aimed at combating obesity and/or diabetes.

### REFERENCES

1. Ailhaud et al., 12 ANNU. REV. NUTR. 207-33 (1992).
2. Aja et al., 291 AM. J. PHYSIOL. REGUL. INTEGR. COMP. PHYSIOL. R148-R154 (2006).
3. Bamshad et al., 275 AM. J. PHYSIOL. R291-R299 (1998).
4. Barbatelli et al., 298 AM. J. PHYSIOL. ENDOCRINOL. METAB. E1244-E1253 (2010).
5. Bartness et al., 275 AM. J. PHYSIOL. R1399-R1411 (1998).
6. Bellinger et al., 76 PHYSIOL. BEHAV. 431-42 (2002).
7. Bewick et al., 19 FASEB J. 1680-82 (2005).
8. Bi et al., 281 AM. J. PHYSIOL. REGUL. INTEGR. COMP. PHYSIOL. R254-R260 (2001).
9. Bi et al., 285 AM. J. PHYSIOL. REGUL. INTEGR. COMP. PHYSIOL. R1030-R1036 (2003).
10. BILLINGTON ET AL., 260 AM. J. PHYSIOL. R321-R327 (1991).
11. Cannon et al., 84 PHYSIOL. REV. 277-359 (2004).
12. Cinti et al., 297(5) AM. J. PHYSIOL. ENDOCRINOL. METAB. E977-E986 (2009).
13. Clark et al., 115 ENDOCRINOLOGY 427-29 (1984).
14. Cone et al., 27 ENDOCR. REV. 736-49 (2006).
15. Cypess et al., 360 N. ENGL. J. MED. 1509-17 (2009).
16. Dimicco et al., 292 AM. J. PHYSIOL. REGUL. INTEGR. COMP. PHYSIOL. R47-R63 (2007).
17. Egawa et al., 260 AM. J. PHYSIOL. R328-R334 (1991).
18. Elmquist et al., 22 NEURON 221-32 (1999).
19. Fredholm et al., 80 ACTA PHYSIOL. SCAND. 567-76 (1970).
20. Gardiner et al., 327 BIOCHEM. BIOPHYS. RES. COMMUN. 1088-93 (2005).
21. Gooley et al., 9 NAT. NEUROSCI. 398-407 (2006).
22. Gropp et al., 8 NAT. NEUROSCI. 1289-91 (2005).
23. Guan et al., 59 BRAIN RES. MOL. BRAIN RES. 273-79 (1998).
24. Handschin et al., 27 ENDOCR. REV. 728-35 (2006).
25. Himms-Hagen et al., 266 AM. J. PHYSIOL. R13710R1382 (1994).
26. Jimenez et al., 270 EUR. J. BIOCHEM. 699-705 (2003).
27. Kawaguchi et al., 288 AM. J. PHYSIOL. REGUL. INTEGR. COMP. PHYSIOL. R1800-R1805 (2005).
28. Kesterson et al., 11 MOL. ENDOCTRINOL. 630-37 (1997).
29. Landry et al., 290 AM. J. PHYSIOL. REGUL. INTEGR. COMP. PHYSIOL. R1527-R1534 (2006).
30. Levine et al., 5 PEPTIDES 1025-29 (1984).
31. Levine et al., 283 SCIENCE 212-14 (1999).
32. Mieda et al., 103 PROC. NATL. ACAD. SCI. USA 12150-55 (2006).
33. Nagase et al., 97 J. CLIN. INVEST. 2898-2904 (1996).
34. Paxinos, G., and Watson Co. THE RAT BRAIN IN STEREOTAXIC COORDINATES, Fifth Ed. (Elsevier Academic Press, San Diego, Calif. (2005)).
35. Pletnikov et al., 119 BRAIN RES. DEV. BRAIN RES. 179-85 (2000).
36. Rooks et al., 289 AM. J. PHYSIOL. REGUL. INTEGR. COMP. PHYSIOL. R92-R102 (2005).
37. Rosen et al., 4 MOL. CELL 611-17 (1999).
38. Schwartz et al., 404 NATURE 661-71 (2000).
39. Smith et al., 133 ENDOCRINOLOGY 1258-65 (1993).
40. Spiegelman et al., 104 CELL 531-43 (2001).
41. Stanley et al., 35 LIFE SCI. 2635-42 (1984).
42. Tang et al., 322 SCIENCE 583-86 (2008).
43. Tritos et al., 139 ENDOCRINOLOGY 4634-41 (1998).
44. Van Mark Lictenbelt et al., 360 N. ENGL. J. MED. 1500-08 (2009).
45. Virtanen et al., 360 N. ENGL. J. MED. 1518-25 (2009).
46. Weiss et al., 7 NEUROPHARMACOL. 395-403 (1968).
47. Yang et al., 29 J. NEUROSCI. 179-90 (2009).
48. Zaretskaia et al., 928 BRAIN RES. 113-25 (2002).

1. A method for treating obesity comprising administering an effective amount of an agent that inhibits expression of neuropeptide Y (NPY).

2. The method of claim 1, wherein the agent inhibits expression of NPY in the dorsomedial hypothalamus (DMH).

3. The method of claim 1, wherein the agent is selected from the group consisting of a small molecule, a protein, a polypeptide, an RNA interference agent, an antibody, an antisense oligonucleotide, and an enzymatic nucleic acid.

4. The method of claim 3, wherein the RNA interference agent is siRNA.

5. The method of claim 3, wherein the RNA interference agent is shRNA.

6. A recombinant nucleic acid construct comprising a nucleic acid sequence encoding an oligonucleic acid, wherein the oligonucleic acid comprises at least one sequence substantially complementary to at least a part of the neuropeptide Y (NPY) gene or transcript thereof, and wherein the oligonucleic acid inhibits or reduces the expression of NPY.

7. The construct of claim 6, wherein the oligonucleic acid is selected from the group consisting of an antisense oligonucleotide, an RNA interference (RNAi) agent, and an enzymatic nucleic acid.

8. The construct of claim 6, wherein the oligonucleic acid is an RNAi agent.

9. The construct of claim 6, wherein the RNAi agent is a short hairpin RNA.

10. The construct of claim 9, further comprising a U6 promoter operably linked to the nucleic encoding the oligonucleic acid.

11. The construct of claim 6, wherein the oligonucleic acid is substantially complementary to base pairs 257-277 of SEQ ID NO:1.

12. The construct of claim 6, further comprising at least one dorsomedial hypothalamus (DMH)-specific transcription regulating sequence operably linked to the at least one nucleic acid sequence encoding an oligonucleic acid.

13. The construct of claim 12, wherein the hypothalamus-specific transcription regulating sequence is selected from the group consisting of DMH-specific promoters, DMH specific enhancers, and transcription regulating sequences that induce expression in cells of the DMH.

14. The construct of claim 6, wherein the oligonucleic acid is an RNAi agent comprising at least one sequence that is fully complementary to a target sequence of about 20 to about 30 nucleotides of the NPY transcript.

15. A vector comprising the construct of claim 6.

16. The vector of claim 15, wherein the vector is a viral-based vector.

17. The vector of claim 16, where in the viral-based vector is an adenovirus-associated viral-based vector.

18. A host cell comprising the vector of claim 15.

19. A pharmaceutical composition comprising the construct of claim 6.

20. A method for treating an obesity-associated condition or symptom associated therewith in a subject in need thereof comprising administering to the subject an effective amount of an agent that inhibits expression of neuropeptide Y (NPY).

21. The method of claim 20, wherein the obesity-associated condition is selected from the group consisting of cardiovascular disease, type 2 diabetes, cancer, steatohepatitis, and osteoarthritis.

22. A method for reducing fat cell mass in a subject in need thereof comprising administering to the subject an effective amount of an agent that inhibits expression of neuropeptide Y (NPY).

23. A method for treating obesity in a subject comprising administering an agent that transforms white adipose tissue into brown adipose tissue in the subject thereby treating obesity.

24. The method of claim 23, wherein the agent comprises an agent that inhibits NPY expression.

25. The method of claim 24, wherein the agent inhibits NPY expression in the DMH.

26. The method of any of claim 22, wherein the agent comprises a recombinant nucleic acid construct comprising a nucleic acid sequence encoding an oligonucleic acid comprising at least one sequence substantially complementary to at least a part of the neuropeptide Y (NPY) gene or transcript thereof, and wherein the oligonucleic acid inhibits expression of NPY.

27. The method of claim 26, wherein the recombinant nucleic acid construct is administered locally to specific fat depots or is administered systemically.


SEQUENCE LISTING (16 sequences)

SEQ ID NO: 1 - DNA, 576 bp, Homo sapiens
  misc_feature: Human neuropeptide Y mRNA
gcaccccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattcaaaagtgaaaaaaaaaaaaaaaa

SEQ ID NO: 2 - DNA, 21 bp, Homo sapiens
  misc_feature: Base pairs 257-277 of human NPY mRNA (siRNA target sequence)
aacctcatcaccaggcagaga

SEQ ID NO: 3 - DNA, 53 bp, Rattus norvegicus
  misc_feature: shRNA sequence (double strands, see SEQ ID NO4) for generating a recombinant shRNA expression vector using the U6 promoter
tctcatcaccagacagagattcaagagatctctgtctggtgatgagatttttt

SEQ ID NO: 4 - DNA, 61 bp, Rattus norvegicus
  misc_feature: shRNA sequence (double strands, see SEQ ID NO3) for generating a recombinant shRNA expression vector using the U6 promoter
aattaaaaaatctcatcaccagacagagatctcttgaatctctgtctggtgatgagaggcc

SEQ ID NO: 5 - DNA, 21 bp, Rattus norvegicus
  misc_feature: Base pairs 240-260 of rat NPY mRNA (siRNA target sequence)
aatctcatcaccagacagaga

SEQ ID NO: 6 - DNA, 539 bp, Rattus norvegicus
  misc_feature: Rat neuropeptide Y mRNA
caagctcattcctcgcagaggcgcccagagcagagcacccgctgcgcagagaccacagcccgcccgccatgatgctaggtaacaaacgaatggggctgtgtggactgaccctcgctctatccctgctcgtgtgtttgggcattctggctgaggggtacccctccaagccggacaatccgggcgaggacgcgccagcagaggacatggccagatactactccgctctgcgacactacatcaatctcatcaccagacagagatatggcaagagatccagccctgagacactgatttcagatctcttaatgagagaaagcacagaaaatgcccccagaacaaggcttgaagacccttccatgtggtgatgggaaatgaaacttgctctcctgacttttcctagtttccccccacatctcatctcatcctgtgaaaccagtctgcctgtcccacccaatgcatgccaccaccaggctggattccgacccatttcccttgttgtcgttgtatatatgtgtgtttaaataaagtatcatgcattc

SEQ ID NO: 7 - DNA, 23 bp, Artificial Sequence
  UCP1 forward primer
cgttccaggatccgagtcgcaga

SEQ ID NO: 8 - DNA, 23 bp, Artificial Sequence
  UCP1 reverse primer
tcagctcttgtcgccgggttttg

SEQ ID NO: 9 - DNA, 20 bp, Artificial Sequence
  PGC1a forward primer
aatgcagcggtcttagcact

SEQ ID NO: 10 - DNA, 20 bp, Artificial Sequence
  PGC1a reverse primer
gtgtgaggagggtcatcgtt

SEQ ID NO: 11 - DNA, 23 bp, Artificial Sequence
  PPAR-gamma forward primer
gcctgcggaagccctttggtgac

SEQ ID NO: 12 - DNA, 23 bp, Artificial Sequence
  PPAR-gamma reverse primer
ttggcgaacagctgggaggactc

SEQ ID NO: 13 - DNA, 20 bp, Artificial Sequence
  FAS forward primer
tcgagacacatcgtttgagc

SEQ ID NO: 14 - DNA, 20 bp, Artificial Sequence
  FAS reverse primer
tcaaaaagtgcatccagcag

SEQ ID NO: 15 - DNA, 23 bp, Artificial Sequence
  CPT1a forward primer
ggcagaagagatggcggtcgatg

SEQ ID NO: 16 - DNA, 23 bp, Artificial Sequence
  CPT1a reverse primer
ccccaagtcaacggcagagcaga